(19) United States
(12) Patent Application Publication
(10) Pub. No.: US 2010/0151388 A1
(43) Pub. Date: Jun. 17, 2010

(54) (Meth)acrylate compound, photosensitive polymer, and resist composition including the same

(76) Inventors: Young-Soo Yang, Uiwang-si (KR); Seung-Jib Choi, Uiwang-si (KR); Jun-Sunk Kim, Uiwang-si (KR); Sang-Jun Choi, Uiwang-si (KR)

Correspondence Address: LEE & MORSE, P.C., 3141 FAIRVIEW PARK DRIVE, SUITE 500, FALLS CHURCH, VA, 22042, US

(21) Appl. No.: 12/654,135

(22) Filed: Dec. 11, 2009

(30) Foreign Application Priority Data
Dec. 12, 2008 (KR) 10-2008-0126779

(51) Int. Cl. G03F 7/004; C07D 211/60; C08F 226/06; C07D 207/12

(52) U.S. Cl. 430/285.1; 546/245; 548/531; 526/258; 526/265

(57) ABSTRACT

A (meth)acrylate compound having a nitrogen-containing cyclic group, a photosensitive polymer, and a resist composition including the same, the (meth)acrylate compound being represented by the following Chemical Formula 1:

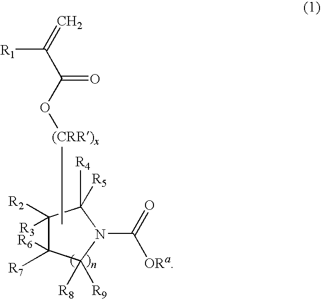

### BACKGROUND

1. Field

Embodiments relate to a (meth)acrylate compound, a photosensitive polymer, and a resist composition including the same.

2. Description of the Related Art

Recently, semiconductor manufacturing processes and integration of semiconductors have increasingly required forming a fine pattern. As a photoresist material, a resist material using a shorter wavelength, e.g., an ArF excimer laser of 193 nm, may be more desirable than a resist material using a conventional KrF excimer laser of 248 nm. However, since a semiconductor device with a capacity of more than 16 gigabytes may require a pattern size of less than about 70 nm according to a design rule, a thickness of a resist film may become thinner. Furthermore, a process margin of underlayer etching has been reduced, and thus, a resist material using an ArF excimer laser may have reached its limit. Reducing a pattern size may cause Line Width Roughness (LWR) and a pattern collapse due to, e.g., reduced adherence between underlayers. A typical ArF resist may include an acryl-based or methacryl-based polymer. Among them, a poly(methacrylate)-based polymer material has been the most commonly used. However, a resist formed from these polymers may exhibit increased LWR due to, e.g., indiscreet acid diffusion. Since acid diffusion may occur from exposure during the semiconductor device manufacturing process if not suitably controlled, it may cause LWR of the materials.

In order to control acid diffusion, a lactone group may be introduced into a polymer. The lactone group may suppress acid diffusion through a hydrogen bond with an acid. However, a mole ratio of the repeating units including a lactone group may not be significantly increased, in order to maintain solubility in a development solution and adhesion to a underlayer.

### SUMMARY

Embodiments are directed to a (meth)acrylate compound, a photosensitive polymer, and a resist composition including the same, which substantially overcome one or more of the problems due to the limitations and disadvantages of the related art.

It is a feature of an embodiment to provide a (meth)acrylate compound including nitrogen-containing cyclic group, which may be prepared at a low cost and may be easily used to prepare a photosensitive polymer.

It is another feature of an embodiment to provide a photosensitive polymer including a repeating unit derived from the (meth)acrylate compound and having reduced LWR and excellent underlayer adhesion when used in a resist.

It is another feature of an embodiment to provide a resist composition including the photosensitive polymer, which may provide excellent lithography performance in a lithographic process using an ultrashort wavelength region, e.g., a 193 nm region and EUV (13.5 nm), as a light source.

At least one of the above and other features and advantages may be realized by providing a (meth)acrylate compound having a nitrogen-containing cyclic group, the (meth)acrylate compound being represented by Chemical Formula 1:

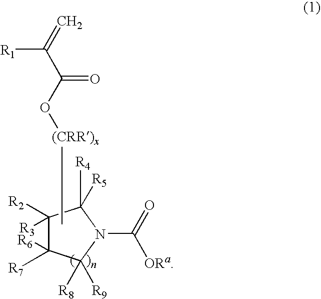

wherein, R₁ is hydrogen or methyl, R₂, R₃, R₄, R₅, R₆, R₇, R₈, and R₉ are each independently hydrogen, a substituted or unsubstituted alkyl, or a substituted or unsubstituted cycloalkyl, R and R′ are each independently hydrogen or a substituted or unsubstituted alkyl, R^{a} is a substituted or unsubstituted alkyl or a substituted or unsubstituted cycloalkyl, x is an integer of 0 to about 3, and n is an integer of about 1 to about 4 and indicates a number of carbons included in the ring of the nitrogen-containing cyclic group.

The (meth)acrylate compound may include at least one compound selected from the group consisting of compounds represented by the following Chemical Formulae 1a to 1f:

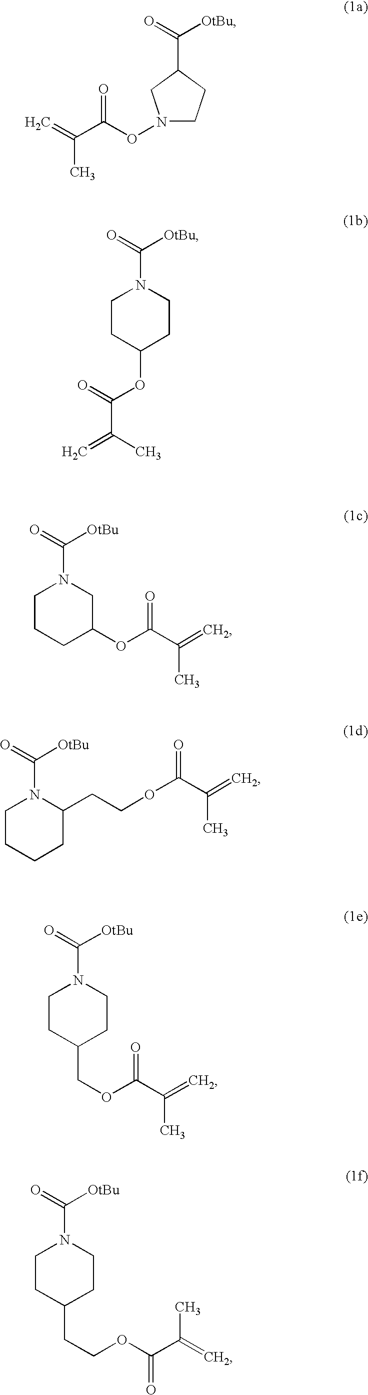

wherein tBu denotes t-butyl.

At least one of the above and other features and advantages may also be realized by providing a photosensitive polymer, comprising repeating units derived from compounds represented by Chemical Formulae 1, 2, 3, and 4:

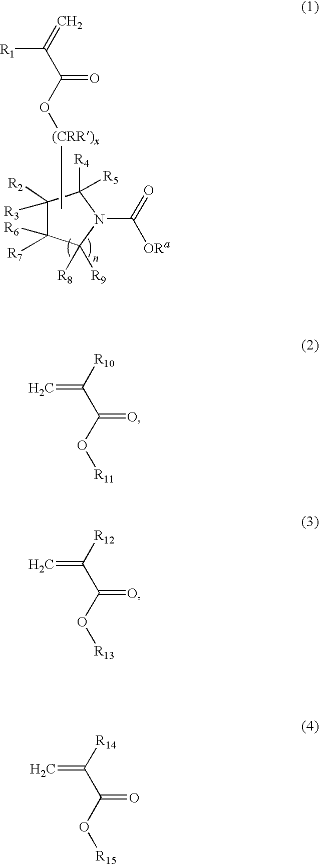

wherein, in Chemical Formula 1 R₁ is hydrogen or methyl, R₂, R₃, R₄, R₅, R₆, R₇, R₈, and R₉ are each independently hydrogen, a substituted or unsubstituted alkyl, or a substituted or unsubstituted cycloalkyl, R and R′ are each independently hydrogen or a substituted or unsubstituted alkyl, R^{a} is a substituted or unsubstituted alkyl or a substituted or unsubstituted cycloalkyl, x is an integer of 0 to about 3, and n is an integer of about 1 to about 4 and indicates a carbon number in the cyclic group, wherein in Chemical Formulae 2-4, R₁₀, R₁₂ and R₁₄ are each independently hydrogen or methyl, in Chemical Formula 2, R₁₁ is a C4 to C20 acid-labile group capable of being decomposed under an acid catalyst, in Chemical Formula 3, R₁₃ is a lactone-derived group, and in Chemical Formula 4, R₁₅ is hydrogen; an alkyl group including a polar functional group, the polar functional group including at least one of a hydroxyl group and a carboxyl group; or a cycloalkyl group including a polar functional group, the polar functional group including at least one of a hydroxyl group and a carboxyl group.

A mole fraction of the repeating unit derived from Chemical Formula 1 may be about 0.01 to about 0.2, a mole fraction of the repeating unit derived from Chemical Formula 2 may be about 0.2 to about 0.5, a mole fraction of the repeating unit derived from Chemical Formula 3 may be about 0.3 to about 0.5, and a mole fraction of the repeating unit derived from Chemical Formula 4 may be about 0.1 to about 0.4, based on a total mole fraction of the repeating units derived from the Chemical Formulae 1, 2, 3, and 4.

The acid labile group may include at least one of norbornyl, isobornyl, cyclodecanyl, adamantyl, norbornyl having a lower alkyl substituent, isobornyl having a lower alkyl substituent, cyclodecanyl having a lower alkyl substituent, adamantly having a lower alkyl substituent, alkoxycarbonyl, alkoxycarbonylalkyl, amyloxycarbonyl, amyloxycarbonylalkyl, 2-tetrahydropyranyloxycarbonyl alkyl, 2-tetrahydrofuranyloxycarbonylalkyl, a tertiary alkyl, and an acetal.

The lactone-derived group may be a group represented by one of Chemical Formula 5 or 6,

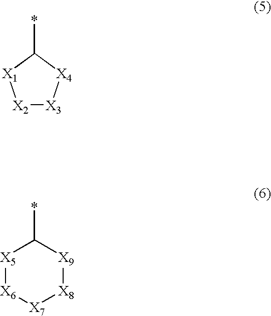

wherein, in Chemical Formula 5, two adjacent groups of X₁ to X₄ are CO and O, respectively, and a remaining two of X₁ to X₄, other than the CO and O, are CR″, wherein R″ is a hydrogen, an alkyl, or an alkylene forming a fused ring with the five-member ring, wherein, in Chemical Formula 6, two adjacent groups of X₅ to X₉ are CO and O, respectively, and a remaining three of X₅ to X₉, other than the CO and O, are one of CR″ or CR′″, R″ being hydrogen, an alkyl, or an alkylene forming a fused ring with the six-member ring, and R′″ being hydrogen, an alkyl, or an ester-containing alkylene forming a fused ring with the six-member ring, at least two R′″ being linked to each other to from a lactone ring.

In Chemical Formula 4, R₁₅ may be 2-hydroxyethyl, 3-hydroxy-1-adamantyl, or 4-hydroxy-2-adamantyl.

The photosensitive polymer may have a weight average molecular weight (Mw) of about 3,000 to about 20,000.

The photosensitive polymer may have a polydispersity (Mw/Mn) of about 1.5 to about 2.5.

At least one of the above and other features and advantages may also be realized by providing a resist composition including (a) a photosensitive polymer according to an embodiment, (b) a photoacid generator (PAG), and (c) a solvent.

The photosensitive polymer may be included in an amount of about 5 to about 15 parts by weight, based on 100 parts by weight of the resist composition.

The photoacid generator may be included in an amount of about 1 to about 15 parts by weight, based on 100 parts by weight of the photosensitive polymer.

The photoacid generator may include at least one of a triarylsulfonium salt, a diaryliodonium salt, and a sulfonate.

The resist composition may further include an organic base, the organic base being included in an amount of about 0.1 to about 1.0 parts by weight, based on 100 parts by weight of the photosensitive polymer.

The organic base may include at least one of triethylamine, triisobutylamine, trioctylamine, triisodecylamine, and triethanolamine.

### DETAILED DESCRIPTION

Korean Patent Application No. 10-2008-0126779, filed on Dec. 12, 2008, in the Korean Intellectual Property Office, and entitled: “(Meth)Acrylate Compound, Photosensitive Polymer, and Resist Composition,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when an element is referred to as being “on” another element, it can be directly on the other element, or intervening elements may also be present. Further, it will be understood that when an element is referred to as being “under” another element, it can be directly under, and one or more intervening elements may also be present. In addition, it will also be understood that when an element is referred to as being “between” two elements, it can be the only element between the two elements, or one or more intervening elements may also be present. Like reference numerals refer to like elements throughout.

As used herein, when specific definition is not otherwise provided, the term “alkyl” refers to a C1 to C10 alkyl, the term “cycloalkyl” refers to a C3 to C10 cycloalkyl, and the term “lower alkyl” refers to a C1 to C4 alkyl. Herein, the alkyl may be a linear or branched alkyl. The term “alkoxy” refers to a C1 to C20 alkoxy, and in one implementation a C1 to C12 alkoxy. The term “alkylene” refers to a C1 to C20 alkylene, and in one implementation, a C1 to C12 alkylene. The term “aryl” refers to a C1 to C20 aryl, and in one implementation a C6 to C12 aryl.

In the present specification, the term “substituted” may refer to at least one hydrogen of a group being substituted with an alkyl or an aryl.

The (meth)acrylate compound according to an embodiment may include a (meth)acrylate compound including a nitrogen-containing cyclic group. According to an embodiment, the (meth)acrylate compound including the nitrogen-containing cyclic group may be represented by the following Chemical Formula 1:

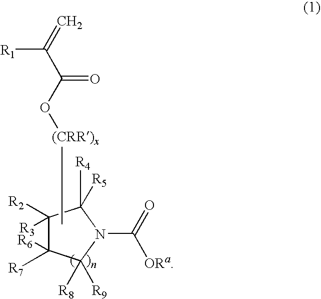

In Chemical Formula 1, R₁ may be, e.g., hydrogen or methyl.

R and R′ may each independently be, e.g., hydrogen or a substituted or unsubstituted alkyl.

x may be an integer of 0 to about 3.

R₂, R₃, R₄, R₅, R₆, R₇, R₈, and R₉ may each independently be, e.g., hydrogen, a substituted or unsubstituted alkyl, or a substituted or unsubstituted cycloalkyl. In addition, one of R₂, R₃, R₄, R₅, R₆, R₇, R₈, and R₉ may be a bond to the CRR′ or the ester oxygen of the (meth)acrylate moiety. In other words, when x is 0, one of R₂, R₃, R₄, R₅, R₆, R₇, R₈, and R₉ may be a bond to the ester oxygen of the (meth)acrylate moiety, i.e., the ester oxygen may be bonded to a ring carbon of the nitrogen containing cyclic group. When x is greater than 0, one of R₂, R₃, R₄, R₅, R₆, R₇, R₈, and R₉ may be a bond to the carbon C of the CRR′, i.e., the carbon C of the CRR′ may be bonded to a ring carbon of the nitrogen containing cyclic group.

R^{a} may be a substituted or unsubstituted alkyl, e.g., t-butyl, or a substituted or unsubstituted cycloalkyl.

n may be an integer of about 1 to about 4 and may indicate a carbon number of the cyclic group. In an implementation, n may be an integer of 1 or 2. In another implementation, the nitrogen-containing cyclic structure may be a pyrrolidine or piperidine structure where n is 1 or 2, respectively. In particular, n may indicate a carbon number of the cyclic group in addition to the three carbons of the cyclic group. For example, when n is 1, the cyclic group may have the pyrrolidine structure and may contain 4 carbons, when n is 2, the cyclic group may have the piperidine structure and may contain 5 carbons, etc.

Specific examples of the (meth)acrylate compound represented by Chemical Formula 1 are represented by Chemical Formulae 1a to 1f:

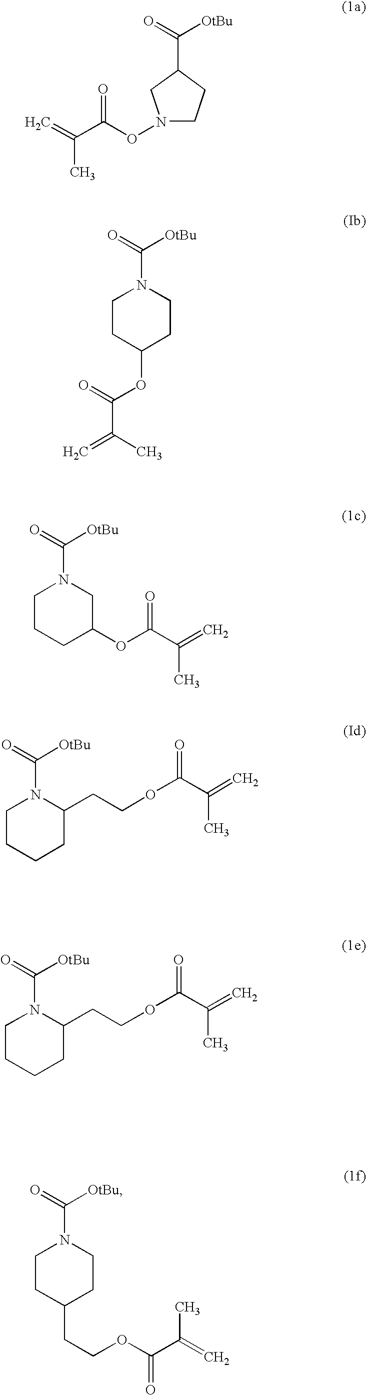

The (meth)acrylate compound having a nitrogen-containing cyclic group represented by Chemical Formula 1 may include one or more compounds represented by the Chemical Formulae 1a to 1f, but are not limited thereto.

The (meth)acrylate compound including a nitrogen-containing cyclic group may be prepared by, e.g., a reaction between nitrogen-containing cyclic compounds including a hydroxyl group and (meth)acryloyl halide e.g., (meth)acryloyl chloride, or (methyl)propenoic anhydride.

A photosensitive polymer including a repeating unit derived from the (meth)acrylate compound of an embodiment may reduce LWR in a resist because, e.g., the (meth)acrylate compound of an embodiment may include an OR^{a} group that is deprotected by an acid, and amine generated in subsequent process may quench acid. Accordingly, the photosensitive polymer may reduce LWR in a resist, so it may be used in preparing a semiconductor device in which a higher resolution is desirable.

The photosensitive polymer of an embodiment may include the repeating unit derived from the (meth)acrylate including the nitrogen-containing cyclic group represented by Chemical Formula 1, as well as repeating units derived from compounds represented by the following Chemical Formulae 2, 3, and 4. The photosensitive polymer may include any type of copolymer without limitation, e.g., a block copolymer including regularly repeated repeating units derived from the compounds represented by Chemical Formulae 1, 2, 3, and 4 and/or a random copolymer including randomly repeated repeating units derived from the compounds represented by Chemical Formulae 1, 2, 3, and 4.

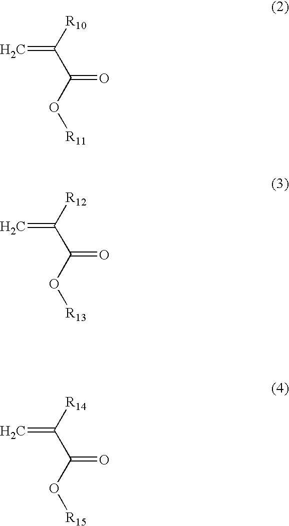

In Chemical Formulae 2-4, R₁₀, R₁₂, and R₁₄ may each independently be hydrogen or methyl.

In Chemical Formula 2, R₁₁ may be, e.g., a C4 to C20 acid-labile group capable of being decomposed under an acid catalyst. In an implementation, R₁₁ may include, e.g., norbornyl, isobornyl, cyclodecanyl, adamantyl, norbornyl having a lower alkyl substituent, isobornyl having a lower alkyl substituent, cyclodecanyl having a lower alkyl substituent, adamantly having a lower alkyl substituent, alkoxycarbonyl, alkoxycarbonyl alkyl, amyloxycarbonyl, amyloxycarbonyl alkyl, 2-tetrahydropyranyloxycarbonyl alkyl, 2-tetrahydrofuranyloxycarbonyl alkyl, a tertiary alkyl, or an acetal. In another implantation, R₁₁ may include, e.g., 2-methyl-2-norbornyl, 2-ethyl-2-norbornyl, 2-methyl-2-isobornyl, 2-ethyl-2-isobornyl, 8-methyl-8-tricyclodecanyl, 8-ethyl-8-tricyclodecanyl, 2-methyl-2-adamantyl, 2-ethyl-2-adamantyl, 2-propyl-2-adamantyl, t-butoxycarbonyl, t-butoxycarbonylmethyl, t-amyloxycarbonyl, t-amyloxycarbonylmethyl, 1-ethoxyethoxycarbonylmethyl, 2-tetrahydropyranyloxycarbonylalkyl, 2-tetrahydrofuranyloxycarbonyl alkyl, a t-butyl, a triethylcarbamyl, 1-methyl cyclohexyl, 1-ethylcyclopentyl, t-amyl, or an acetal.

In Chemical Formula 3, R₁₃ may be, e.g., a lactone-derived group. In an implementation, R₁₃ may be, e.g., a group having a structure represented by at least one of the following Chemical Formula 5 and 6.

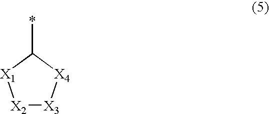

In Chemical Formula 5, two adjacent groups of X₁ to X₄ may be, e.g., CO and O, respectively. The remaining two of X₁ to X₄, other than the CO and O, may be, e.g., CR″, wherein R″ may be, e.g., hydrogen, an alkyl, or an alkylene forming a fused ring with the five-member ring.

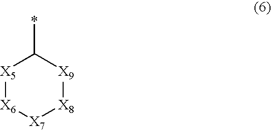

In Chemical Formula 6, two adjacent groups of X₅ to X₉ may be, e.g., CO and O, respectively. In an implementation, the remaining three of X₅ to X₉, other than the CO and O, may be, e.g., CR″, wherein R″ may be, e.g., hydrogen, an alkyl, or an alkylene forming a fused ring with the six-member ring. In another implementation, the remaining three of X₅ to X₉, other than the CO and O, may be, e.g., CR′″, wherein R′″ may be, e.g., hydrogen, an alkyl, an ester-containing alkylene forming a fused ring with the six-member ring, and at least two R′″ may be linked to each other to from a lactone ring.

In an implementation, R₁₃ may be, e.g., butyrolactonyl, valerolactonyl, 1,3-cyclohexanecarbolactonyl, 2,6-norbornanecarbolacton-5-yl, or 7-oxa-2,6-norbornanecarbolacton-5-yl.

In Chemical Formula 4, R₁₅ may be, e.g., hydrogen, an alkyl group including a polar functional group including, e.g., a hydroxyl group and/or carboxyl group, or a cycloalkyl group including a polar functional group including, e.g., a hydroxyl group and/or a carboxyl group. In an implementation, R₁₅ may be, e.g., 2-hydroxyethyl, 3-hydroxy-1-adamantyl, or 4-hydroxy-2-adamantyl.

In an implementation, the photosensitive polymer may be the same as described above and may be a polymer including repeating units derived from compounds represented by Chemical Formulae 1, 2, 3, and 4. Herein, s may indicate a mole fraction of the repeating unit derived from the compound represented by Chemical Formula 1. p may indicate a mole fraction of the repeating unit derived from the compound represented by Chemical Formula 2. q may indicate a mole fraction of the repeating unit derived from the compound represented by Chemical Formula 3. r may indicate a mole fraction of the repeating unit derived from the compound represented by Chemical Formula 4.

In particular, p/(p+q+r+s) may be about 0.2 to about 0.5, q/(p+q+r+s) may be about 0.3 to about 0.5, r/(p+q+r+s) may be about 0.1 to about 0.4, and s/(p+q+r+s) may be about 0.01 to about 0.2. In an implementation, s/(p+q+r+s) may be about 0.03 to about 0.1. In other words, the mole fraction of the repeating units represented by Chemical Formula 1 may be about 0.01 to about 0.2, the mole fraction of the repeating units represented by Chemical Formula 2 may be about 0.2 to about 0.5, the mole fraction of the repeating units represented by Chemical Formula 3 may be about 0.3 to about 0.5, and the mole fraction of the repeating units represented by Chemical Formula 4 may be about 0.1 to about 0.4. In an implementation, the mole fraction of the repeating units represented by Chemical Formula 1 may be about 0.03 to about 0.1.

The photosensitive polymer may have a weight average molecular weight (Mw) of about 3,000 to about 20,000. The photosensitive polymer may have a polydispersity (Mw/Mn) of about 1.5 to about 2.5. Maintaining the polydispersity at about 1.5 to about 2.5 may help ensure that a resist formed using the photosensitive polymer has reduced LWR and excellent resolution.

The photosensitive polymer according to an embodiment may be a copolymer obtained from compounds including a functional nitrogen-containing cyclic group of an embodiment. The photosensitive polymer according to an embodiment may advantageously provide a resist composition for forming a resist having excellent adhesion to an underlayer and reduced LWR. A resist composition including the photosensitive polymer of an embodiment and used in a photolithography process may provide excellent lithography performance.

Another embodiment may provide the resist composition including the photosensitive polymer. In particular, the resist composition may include (a) the photosensitive polymer, (b) a photoacid generator (PAG), and (c) a solvent.

Hereinafter, the components of the resist composition according to an embodiment will be described in more detail.

(a) Photosensitive Polymer

The photosensitive polymer may be the photosensitive polymer of an embodiment, as described above. The photosensitive polymer may be included in the composition in an amount of about 5 to about 15 parts by weight, based on 100 parts by weight of the resist composition. Maintaining the amount of the photosensitive polymer at about 5 to about 15 parts by weight may help ensure that the resist composition has excellent etching resistance and adhesion characteristics.

(b) Photoacid Generator (PAG)

The photoacid generator may include, e.g., an inorganic onium salt and/or an organic sulfonate. In an implementation, the photoacid generator may include, e.g., sulfonate or iodonium salt including at least one of a triarylsulfonium salt, a diaryl iodonium salt, and sulfonate. In another implementation, the photoacid generator may include, e.g., triarylsulfonium triflate, diaryliodonium triflate, triarylsulfonium nonaflate, diaryliodonium nonaflate, succinimidyl triflate, and/or 2,6-dinitrobenzyl sulfonate.

The photoacid generator may be included in the composition in an amount of about 1 to about 15 parts by weight based on 100 parts by weight of the photosensitive polymer. Maintaining the amount of the photoacid generator at about 1 to about 15 parts by weight may help ensure that an exposure dose with respect to the resist composition as well as a transmission of the resist composition may be appropriately controlled.

(c) Solvent

The solvent may include, e.g., propylene glycol monomethyl ether acetate (PGMEA), propylene glycol methyl ether (PGME), ethyl lactate (EL), cyclohexanone, 2-heptanone, and the like.

The solvent may be included as the balance amount of the resist composition without limitation. In an implementation, the solvent may be included in an amount of about 80 parts by weight to about 95 parts by weight, based on 100 parts by weight of the resist composition.

(d) Additive

The resist composition may further include an additive. The additive may include, e.g., an organic base as a quencher in order to control the exposure dose and to form a resist profile.

The organic base may include, e.g., amine-based compounds such as triethylamine, triisobutylamine, trioctylamine, triisodecylamine, and/or triethanolamine.

In an implementation, the organic base may be included in an amount of about 0.1 to about 1 part by weight, based on 100 parts by weight of the photosensitive polymer. Maintaining the amount of the organic base at about 0.1 to about 1 part by weight may help ensure that the exposure dose is not excessively increased, desirable effects may be obtained, and the pattern is well formed.

A process of forming a desirable pattern with the resist composition of an embodiment may be as follows.

A bare silicon wafer or a silicon wafer including an underlayer, e.g., a silicon oxide layer, a silicon nitride layer, or a silicon nitride oxide layer on an upper surface, may be treated with HMDS (hexamethyldisilazane) or an organic anti-reflection coating (bottom anti-reflective coating). Then, the resist composition according to an embodiment may be coated on the silicon wafer at a thickness of about 100 to about 150 nm to provide a resist layer.

The silicon wafer having the resist layer thereon may be soft-baked (i.e., pre-baked) at a temperature of about 90 to about 120° C. for about 60 to about 90 seconds to remove the solvent. Then, the wafer may be exposed to at least one of a variety of exposure light sources, e.g., ArF, EUV (extreme UV), E-beam, and so on. In order to perform a chemical reaction in the exposure region of the resist layer, the wafer may be subjected to PEB (post-exposure baking) at a temperature of about 90 to about 120° C. for about 60 to about 90 seconds.

Then, the resist layer may be developed in a basic aqueous developing solution. The exposure region may have a very high solubility in the basic aqueous developing solution, so it may be easily dissolved and removed during the development. In an implementation, e.g., tetramethylammonium hydroxide (TMAH) may be used as the basic aqueous developing solution. When the exposure light source is an ArF excimer laser, an about 80 to about 100 nm line and space pattern may be obtained at a dose of about 5 to about 50 mJ/cm².

The resist pattern obtained from the above process may be used as a mask. The underlayer, e.g., a silicon oxide layer, may be etched by using a certain etching gas, e.g., a plasma of halogen gas or fluorocarbon gas such as CF₄. Resist pattern that remains on the wafer may be removed by using a stripper to provide a desired silicon oxide layer pattern.

The following Examples are suggested for helping in understanding of the embodiments, but the embodiments are not limited to the following examples.

### Preparation Example 1

### Tert-butyl-4-(methacryloyloxy)piperidine-1-carboxylate salt

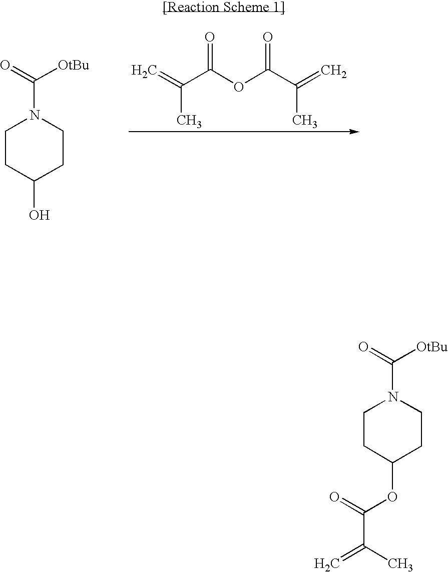

According to the process as shown in Reaction Scheme 1, a tert-butyl-4-methacryloyloxypiperidine-1-carboxylate salt was synthesized.

In particular, 25.4 g of tert-butyl-4-hydroxypiperidine-1-carboxylate salt and 0.771 g of 4-dimethylaminopyridine were dissolved in 170 mL of methylene chloride. Then, 30.8 mL of diisopropylethylamine and 22.6 mL of methyl-propenoic anhydride were added thereto. The resulting product was reacted at a room temperature for about 15 hours.

After the reaction was complete, the reactant was diluted in an excess of ethyl acetate, and the diluted product was rinsed with a saturated sodium bicarbonate solution and a saturated sodium chloride solution. Then, a predetermined amount of sodium sulfate was added to the rinsed product and was agitated for about 15 minutes. The agitated product was filtered to remove sodium sulfate, and the solvent was removed from the resulting material under a low pressure condition to extract a product (I), and purified with a column chromatography (hexane:ethyl acetate=10:1 volume ratio) (yield: 90%).

¹H-NMR (CDCl₃, ppm): 6.1 (s, 1H, vinyl), 5.6 (s, 1H, vinyl),

5.0 (tt, 1H, O—CH—), 3.7 (m, 2H, N—CH₂—), 3.4 (m, 2H, N—CH₂—),

1.9 (s, 3H, —CH₃), 1.8 (m, 2H, —CH₂—), 1.7 (m, 2H, —CH₂—),

1.4 (s, 9H, -tBu)

### Preparation Example 2

### Synthesis of Photosensitive Polymer

3 mmol of the tert-butyl-4-(methacryloyloxy)piperidine-1-carboxylate salt synthesized according to Preparation Example 1, 35 mmol of γ-butyrolactonyl methacrylate (GBLMA), 35 mmol of 2-methyl-2-adamantyl methacrylate (MAMA) and 30 mmol of 3-hydroxy-1-adamantyl methacrylate (HAMA) were put in a flask and dissolved with propyleneglycol monomethyl ether acetate (PGMEA) solvent in a 3:1 weight ratio of solvent to the total weight of monomers therein. Then, 15 mmol of dimethyl-2,2′-azobis(2-methylpropinonate) (V601, Wako Pure Chemical Industries Ltd.) was added thereto as a polymerization initiator. The mixture solution was polymerized at a temperature of 80° C. for 4 hours.

When the polymerization was complete, the reactant was slowly precipitated in an excess amount of a hexane solvent. The precipitate was filtered and dissolved in an appropriate amount of dioxane. The solution was then re-precipitated in methanol. Then, the precipitate was dried in a 50° C. vacuum oven for 24 hours, obtaining a photosensitive polymer with repeating units represented by the following Chemical Formulae 7a, 7b, 7c, and 7d (yield: 51%). The photosensitive polymer had a weight average molecular weight (Mw) of 10,659 and polydispersity (Mw/Mn) of 1.41. When the number of moles of the repeating units represented by the following Chemical Formulae 7a, 7b, 7c, and 7d present in the polymer were p, q, r and s respectively, p=35, q=35, r=30, and s=3.

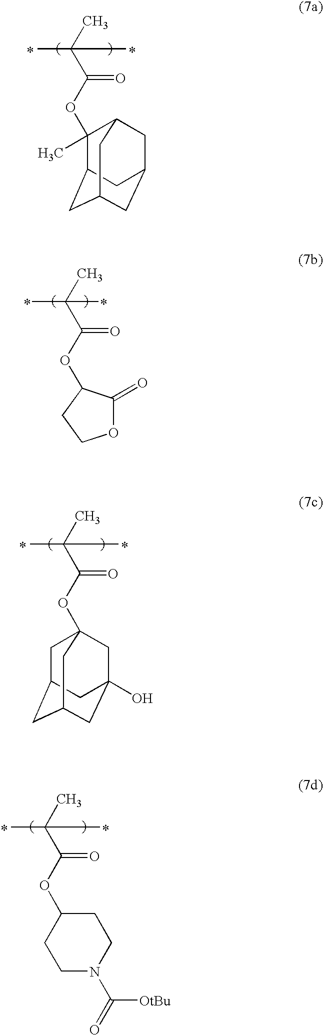

### Preparation Example 3

### Preparation of a Resist Composition and Lithography Performance

A resist composition was prepared by completely dissolving 0.8 g of the photosensitive polymer according to Preparation Example 2 and 0.02 g of a triphenylsulfonium nonaflate photoacid generator in 17 g of propyleneglycol monomethyl etheracetate/ethyl lactate (6/4 volume ratio) and then, completely dissolving 1 mg of triethanol amine, as an organic base.

### Experimental Example 1

### Evaluation of Resolution and LWR

The resist composition of Preparation Example 3 was filtered by using a 0.1 μm thick membrane filter. The filtered resist composition was coated to a thickness of 140 nm on a silicon wafer and soft-baked (SB) at a temperature of 110° C. for 60 seconds, the silicon wafer having been treated to have 600 Å thickness with an organic BARC (AR46, Rhom & Hass Company). The coated wafer was exposed to light with an ArF scanner (0.78 NA, dipole), post-exposure baked (PEB), and then, developed in a 2.38 wt % tetramethylammonium hydroxide aqueous solution for 60 seconds.

As a result, a 90 nm line and space pattern was obtained. Herein, LWR was 6 nm. The LWR was measured using in-line SEM (S-9200; Hitachi).

### Preparation Example 4

### Tert-butyl-3-(methacryloyloxy)pyrrolidine-1-carboxylate salt

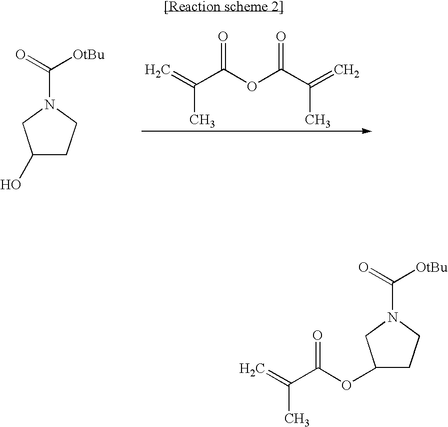

According to the method as shown in Reaction Scheme 2, a tert-butyl-3-(methacryloyloxy)piperidine-1-carboxylate salt was synthesized.

In particular, 25.0 g of tert-butyl-3-hydroxypyrrolidine-1-carboxylate salt and 0.816 g of 4-dimethylaminopyridine were dissolved in 180 mL of methylene chloride, and 32.6 mL of diisopropylethylamine and 23.9 mL of methyl-propenoic anhydride were added thereto. The resulting product was reacted at a room temperature for about 15 hours.

When the reaction was complete, the reactant was diluted in an excess of ethyl acetate, and the diluted product was rinsed with a saturated sodium bicarbonate solution and a saturated sodium chloride solution. Then, a predetermined amount of sodium sulfate was added into the rinsed product and was agitated for about 15 minutes. The agitated product was filtered to remove sodium sulfate, and the reactant solvent was removed under a low pressure condition to extract a product (I), and purified with a column chromatography (hexane:ethyl acetate=8:1 volume ratio) (yield: 91%).

¹H-NMR (CDCl₃, ppm): 6.3 (s, 1H, vinyl), 5.8 (s, 1H, vinyl),

4.1 (tt, 1H, O—CH—), 3.7 (m, 2H, N—CH₂—), 3.4 (m, 2H, N—CH₂—),

2.0 (s, 3H, —CH₃), 1.9 (m, 1H, —CH₂—), 1.7 (m, 1H, —CH₂—),

1.4 (s, 9H, -tBu)

### Preparation Example 5

### Synthesis of Photosensitive Polymer

3 mmol of the tert-butyl-3-(methacryloyloxy)piperidine-1-carboxylate salt synthesized according to Preparation Example 4, 35 mmol of γ-butyrolactonyl methacrylate (GBLMA), 35 mmol of 2-methyl-2-adamantyl methacrylate (MAMA) and 30 mmol of 3-hydroxy-1-adamantyl methacrylate (HAMA) were put in a flask and dissolved with propyleneglycol monomethyl ether acetate (PGMEA) solvent in a 3:1 weight ratio of solvent to the total weight of the monomers therein. Then, 15 mmol of dimethyl-2,2′-azobis(2-methylpropionate) (V601, Wako Pure Chemical Industries Ltd.) was added thereto as a polymerization initiator. The mixture solution was polymerized at a temperature of 80° C. for 4 hours.

When the polymerization was complete, the reactant was slowly precipitated in an excess of a hexane solvent. The precipitate was filtered and dissolved in an appropriate amount of dioxane. Then, the solution was re-precipitated in methanol. Then, the precipitate was dried in a 50° C. vacuum oven for 24 hours, obtaining a photosensitive polymer with repeating units represented by the following Chemical Formulae 8a, 8b, 8c, and 8d (yield: 55%). The photosensitive polymer had a weight average molecular weight (Mw) of 10,923 and polydispersity (Mw/Mn) of 1.33. When the number of moles of the repeating units represented by the following Chemical Formulae 8a, 8b, 8c, and 8d in the polymer were p, q, r and s respectively, p=35, q=35, r=30, and s=3.

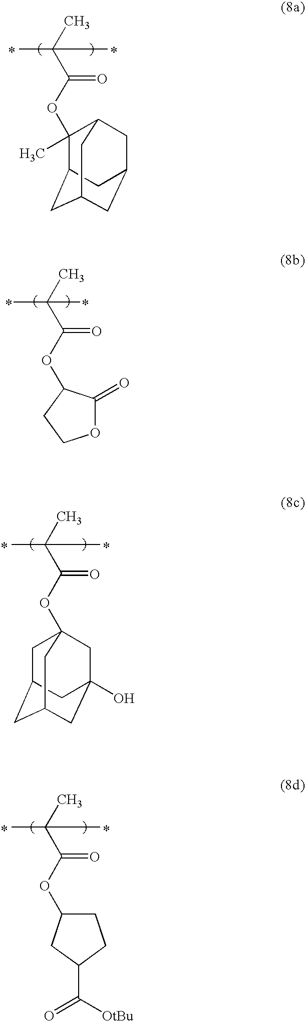

### Preparation Example 6

### Preparation of a Resist Composition and Lithography Performance

A resist composition was prepared by completely dissolving 0.8 g of the photosensitive polymer according to Preparation Example 5 and 0.02 g of a triphenyl sulfonium nonaflate photoacid generator in 17 g of propylene glycol monomethyl etheracetate/ethyl lactate (6/4) and then, dissolving 1 mg of triethanol amine, as an organic base.

### Experimental Example 2

### Evaluation of Resolution and LWR

The resist composition of Preparation Example 6 was filtered by using a 0.1 μm thick membrane filter. The filtered resist composition was coated to a thickness of 140 nm on a silicon wafer, the wafer having been treated to have 600 Å thickness with an organic BARC (AR46, Rhom & Hass Company), and soft-baked (SB) at a temperature of 110° C. for 60 seconds. The coated wafer was exposed to light with an ArF scanner (0.78 NA, dipole), post-exposure baked (PEB), and then, developed in a 2.38 wt % tetramethylammonium hydroxide aqueous solution for 60 seconds.

As a result, a 90 nm line and space pattern was obtained. Herein, LWR was 5 nm. The LWR was measured using in-line SEM (S-9200; Hitachi).

The photosensitive polymer including a repeating unit derived from a (meth)acrylate compound including a nitrogen-containing cyclic group may reduce LWR in a resist by controlling acid diffusion. In addition, the presence of the nitrogen-containing cyclic group may impart the resist with excellent adherence to the underlayers and reduced LWR during the lithography process. Accordingly, the photosensitive polymer of an embodiment may be used to prepare a chemically-amplified resist composition.

In addition, the resist composition obtained using the photosensitive polymer may exhibit reduced LWR and may have an excellent adherence between the underlayers. As a result, it may exhibit reduced pattern collapse during preparation of a semiconductor device. Therefore, it may be advantageously used to fabricate a next generation semiconductor device.

Exemplary embodiments have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A (meth)acrylate compound having a nitrogen-containing cyclic group, the (meth)acrylate compound being represented by the following Chemical Formula 1: 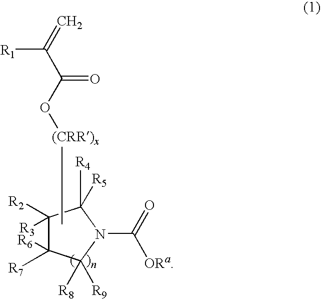
wherein, R₁ is hydrogen or methyl,
R₂, R₃, R₄, R₅, R₆, R₇, R₈, and R₉ are each independently hydrogen, a substituted or unsubstituted alkyl, or a substituted or unsubstituted cycloalkyl,
R and R′ are each independently hydrogen or a substituted or unsubstituted alkyl,
R^{a} is a substituted or unsubstituted alkyl or a substituted or unsubstituted cycloalkyl,
x is an integer of 0 to about 3, and
n is an integer of about 1 to about 4 and indicates a number of carbons included in the ring of the nitrogen-containing cyclic group.

2. The (meth)acrylate compound as claimed in claim 1, wherein the (meth)acrylate compound includes at least one compound selected from the group consisting of compounds represented by the following Chemical Formulae 1a to 1f: 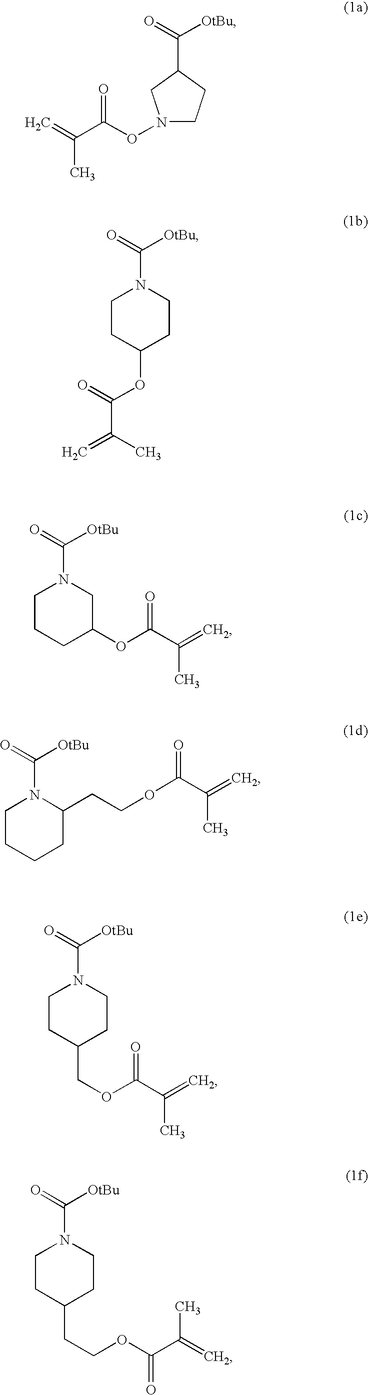
wherein tBu denotes t-butyl.

3. A photosensitive polymer, comprising repeating units derived from compounds represented by Chemical Formulae 1, 2, 3, and 4: 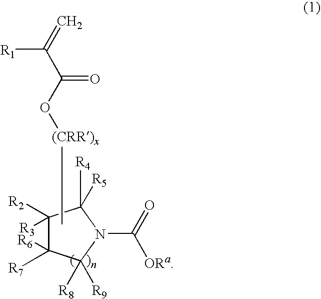
wherein, in Chemical Formula 1:
R₁ is hydrogen or methyl,
R₂, R₃, R₄, R₅, R₆, R₇, R₈, and R₉ are each independently hydrogen, a substituted or unsubstituted alkyl, or a substituted or unsubstituted cycloalkyl,
R and R′ are each independently hydrogen or a substituted or unsubstituted alkyl,
R^{a} is a substituted or unsubstituted alkyl or a substituted or unsubstituted cycloalkyl,
x is an integer of 0 to about 3, and
n is an integer of about 1 to about 4 and indicates a carbon number in the cyclic group, 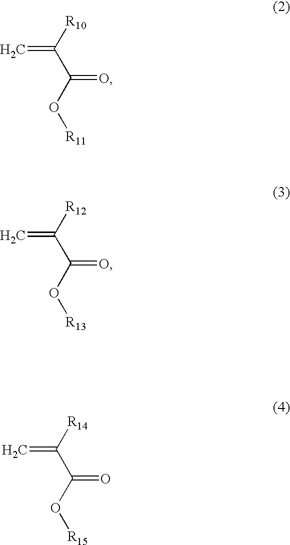
wherein:
in Chemical Formulae 2-4, R₁₀, R₁₂ and R₁₄ are each independently hydrogen or methyl,
in Chemical Formula 2, R₁₁ is a C4 to C20 acid-labile group capable of being decomposed under an acid catalyst,
in Chemical Formula 3, R₁₃ is a lactone-derived group, and
in Chemical Formula 4, R₁₅ is hydrogen; an alkyl group including a polar functional group, the polar functional group including at least one of a hydroxyl group and a carboxyl group; or a cycloalkyl group including a polar functional group, the polar functional group including at least one of a hydroxyl group and a carboxyl group.

4. The photosensitive polymer as claimed in claim 3, wherein:
a mole fraction of the repeating unit derived from Chemical Formula 1 is about 0.01 to about 0.2,
a mole fraction of the repeating unit derived from Chemical Formula 2 is about 0.2 to about 0.5,
a mole fraction of the repeating unit derived from Chemical Formula 3 is about 0.3 to about 0.5, and
a mole fraction of the repeating unit derived from Chemical Formula 4 is about 0.1 to about 0.4, based on a total mole fraction of the repeating units derived from the Chemical Formulae 1, 2, 3, and 4.

5. The photosensitive polymer as claimed in claim 3, wherein the acid labile group includes at least one of norbornyl, isobornyl, cyclodecanyl, adamantyl, norbornyl having a lower alkyl substituent, isobornyl having a lower alkyl substituent, cyclodecanyl having a lower alkyl substituent, adamantly having a lower alkyl substituent, alkoxycarbonyl, alkoxycarbonylalkyl, amyloxycarbonyl, amyloxycarbonylalkyl, 2-tetrahydropyranyloxycarbonyl alkyl, 2-tetrahydrofuranyloxycarbonylalkyl, a tertiary alkyl, and an acetal.

6. The photosensitive polymer as claimed in claim 3, wherein the lactone-derived group is a group represented by one of Chemical Formula 5 or 6: 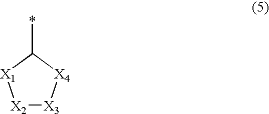
wherein, in Chemical Formula 5, two adjacent groups of X₁ to X₄ are CO and O, respectively, and a remaining two of X₁ to X₄, other than the CO and O, are CR″, wherein R″ is a hydrogen, an alkyl, or an alkylene forming a fused ring with the five-member ring, 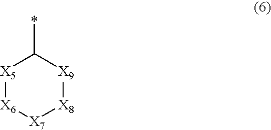
wherein, in Chemical Formula 6, two adjacent groups of X₅ to X₉ are CO and O, respectively, and a remaining three of X₅ to X₉, other than the CO and O, are one of CR″ or CR′″, R″ being hydrogen, an alkyl, or an alkylene forming a fused ring with the six-member ring, and R′″ being hydrogen, an alkyl, or an ester-containing alkylene forming a fused ring with the six-member ring, at least two R′″ being linked to each other to from a lactone ring.

7. The photosensitive polymer as claimed in claim 3, wherein, in Chemical Formula 4, R₁₅ is 2-hydroxyethyl, 3-hydroxy-1-adamantyl, or 4-hydroxy-2-adamantyl.

8. The photosensitive polymer as claimed in claim 3, wherein the photosensitive polymer has a weight average molecular weight (Mw) of about 3,000 to about 20,000.

9. The photosensitive polymer as claimed in claim 3, wherein the photosensitive polymer has a polydispersity (Mw/Mn) of about 1.5 to about 2.5.

10. A resist composition, comprising
(a) a photosensitive polymer according to claim 3;
(b) a photoacid generator (PAG); and
(c) a solvent.

11. The resist composition as claimed in claim 10, wherein the photosensitive polymer is included in an amount of about 5 to about 15 parts by weight, based on 100 parts by weight of the resist composition.

12. The resist composition as claimed in claim 10, wherein the photoacid generator is included in an amount of about 1 to about 15 parts by weight, based on 100 parts by weight of the photosensitive polymer.

13. The resist composition as claimed in claim 10, wherein the photoacid generator includes at least one of a triarylsulfonium salt, a diaryliodonium salt, and a sulfonate.

14. The resist composition as claimed in claim 10, further comprising:
an organic base, the organic base being included in an amount of about 0.1 to about 1.0 parts by weight, based on 100 parts by weight of the photosensitive polymer.

15. The resist composition as claimed in claim 14, wherein the organic base includes at least one of triethylamine, triisobutylamine, trioctylamine, triisodecylamine, and triethanolamine.
